(19) United States
(12) Patent Application Publication
(10) Pub. No.: US 2016/0113841 A1
(43) Pub. Date: Apr. 28, 2016

(54) VIBRATING MASSAGE ROLLER WITH AMPLITUDE ADJUSTMENT

(71) Applicant: Health e Vibrations, LLC, Salt Lake City, UT (US)

(72) Inventors: Brian P. Godfrey, Salt Lake City, UT (US); Gregory R. Mathy, San Diego, CA (US); Theodore B. Hill, San Diego, CA (US); Thomas A. Deiters, San Diego, CA (US)

(21) Appl. No.: 14/924,035

(22) Filed: Oct. 27, 2015

Related U.S. Application Data
(60) Provisional application No. 62/069,053, filed on Oct. 27, 2014.

(51) Int. Cl. A61H 23/02; A61F 7/00; A61F 7/10; A61H 15/00

(52) U.S. Cl. CPC A61H 23/0263; A61H 15/0092; A61F 7/007; A61F 7/10; A61H 2015/0014; A61F 2007/0071; A61F 2007/108

(57) ABSTRACT

A process of massaging a subject is provided that includes the provision of a vibratory massage roller with a user controlled magnitude of vibratory amplitude having a casing with a casing surface extending over a length of the vibratory roller. A series of weights inside the casing may be positioned by the user to vary and control the amplitude of the vibration produced by the roller. A motor is included in the housing and is electrically coupled to a power source. The power source is energized to induce a vibratory amplitude to the surface that varies less than thirty amplitude percent across the central seventy percent of the length. The massage roller is contacted with the subject to massage the subject.

### CROSS-REFERENCE TO RELATED APPLICATION

This application claims priority of U.S. Provisional Patent Application Ser. No. 62/069,053 filed Oct. 27, 2014, which is incorporated herein by reference.

### FIELD OF THE INVENTION

The present invention relates to the field of massagers and more particularly relates to a massage roller that affords a level of uniformity of vibration with vibration amplitude adjustment to a subject along the length of the roller.

### BACKGROUND OF THE INVENTION

Massage is the manipulation of subject muscles and soft tissues in order to affect a release of tension. Massage involves many strategies, including kneading and stretching muscles, percussive striking, and vibration. Massage is well known in the fields of medicine, chiropractic, physical therapy, kinesiotherapy and fitness. Massage is practiced universally around the globe, both professionally and personally, and is recognized as providing some benefits to the mental, emotional, and physical health of those receiving it.

In light of the almost universal appeal and recognition of massage, many different styles of massage have been developed, ranging from Shiatsu, Swedish, Deep Tissue, and others, and tools to aid in massage have developed in each discipline. One such tool is known as a massage roller or foam roller. Foam Rollers are cylindrical or semi-cylindrical bodies made of a compliant material, such as foam, which are used for massaging and stretching soft tissues, increasing circulation, reducing pain, tension and stress from the soft tissues, improving posture and alignment, increase spinal mobility. Core and corrective exercises can be used with the roller as well.

Prior art solutions that use eccentric weights rotating about an axle fail to evenly distribute vibrations to a roller or other massager surface as they generally have only two contact points, one at the motor and one at or by a terminal end of the axle. This localizes all vibrational transmission at those two end points and can cause “weak spots” or “dead zones” along the length of a massager apparatus, especially if they are improperly spaced and allow destructive interference between the two points of contact. This spatial disparity in vibrational amplitude is a result of construction method with a motor and an eccentric weight mounted to a shaft extending from the motor to define a cylindrical axis. Stronger vibrations are created proximal to the weight with the vibrational amplitude decaying as a functional of lateral distance from the eccentric weight. As a result, when the weight is at one end of the roller, vibrations will dissipate inward from the weighted end along the length of the roller, whereas, a centrally located weight has vibrations that will still dissipate towards the ends. This vibrational amplitude decay is especially noticeable in longer rollers. Furthermore, the prior art solutions typically do not allow the user to adjust the amplitude of the vibration of the message roller.

The use of several motors along a roller coupled to eccentric weights to alleviate these problems has met with limited success and also creates harmonic vibrating waves that tend to create points of low amplitude strength vibrations at certain positions along the roller with the position in part dictated by the rotational rate. Exemplary of these efforts is U.S. Pat. No. 6,647,572 in which vibratory nodes are partially mitigated and may be more evenly distributed, yet the reliance on several motors increases cost, weight, and device proclivity to breakage. The problems posed by motor failure are enhanced by the fact that the motor is generally inaccessible for repair and effectively encased in a surrounding roller material. Still another attempt to address the problem of vibrational amplitude inhomogeneity along the length of the roller involves mounting multiple eccentric weights along a single rotating shaft; however, this tends to either accentuate the inhomogeneity if the weights are radially aligned or if radially distributed around the shaft, torque is imparted to the motor that leads to motor damage.

Thus, there exists a need for a vibrating massage roller that provides a degree of positional uniformity in vibration along the roller imparted to a subject in contact with the roller, while also allowing the user to adjust the amplitude of the vibration.

### SUMMARY OF THE INVENTION

A process of massaging a subject is provided that includes the provision of a vibratory massage roller having a casing with a casing surface extending over a length of the vibratory roller. The configuring of one or more weights within the casing, where the positions of the one or more weights in relation to a rotating shaft concentric to the casing and driven by a motor determines a magnitude of a vibratory amplitude. The motor is included in the housing and is electrically coupled to a power source. The power source is energized to induce a vibratory amplitude to the surface that varies less than 30 amplitude percent across the central 70 percent of the length. The massage roller is contacted with the subject to massage the subject.

A vibratory massage roller is provided with a casing having a surface extending over a length of the vibratory roller, a motor in the housing, the motor electrically coupled to a power source. One or more weights within the casing, where the configuration of the positions of the one or more weights in relation to a rotating shaft concentric to the casing and driven by the motor determines a magnitude of a vibratory amplitude; and wherein the power source induces a vibratory amplitude to the surface that varies less than thirty amplitude percent across the central seventy percent of the length.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a perspective view of a massage roller according to one embodiment of the invention;

FIG. 2 is a perspective view of the massage roller of FIG. 1, with an optional covering sleeve;

FIG. 3 is a front elevation of the massage roller of FIG. 2;

FIG. 4 is a sectional view of the massage roller of FIG. 3, taken along line A-A;

FIG. 5A is a sectional view of a massage roller where the vibrational amplitude is controlled via a flexible cable that changes the eccentricity of the weights, along the same line as FIG. 4;

FIG. 5B is a detail view of FIG. 5A showing the flexible cable feeding though the hollow portion of the shaft toward the weights according to an embodiment of the invention;

FIG. 6 is a sectional view of a massage roller according to a further embodiment of FIG. 5A, where two separate adjustable cables are used to change the eccentricity of the weights from opposing sides of the message roller according to embodiments of the invention;

FIG. 7A is a sectional view of a massage roller where the vibrational amplitude is controlled via an umbrella mechanism flexible that changes the eccentricity of the weights, along the same line as FIG. 4;

FIGS. 7B and 7C is a detail view of FIG. 7A showing a detailed view of the umbrella mechanism with the weights in a before and after full deployment for maximum vibrational amplitude, respectively according to an embodiment of the invention;

FIG. 8A is a sectional view of a massage roller where the vibrational amplitude is controlled via magnets that control the radial distribution of the total weight that changes the eccentricity of the weights, along the same line as FIG. 4;

FIG. 8B is a detail view of the magnet and sliding weight according to an embodiment of the invention;

FIG. 9 is a sectional view of a massage roller where the vibrational amplitude is controlled via the manual addition of weight to the eccentricity of the weights, along the same line as FIG. 4;

FIG. 10A is a sectional view of a massage roller where the vibrational amplitude is controlled via an overrunning bearing that change the total magnitude of weight, along the same line as FIG. 4;

FIG. 10B is a detail view of the overrunning bearing according to an embodiment of the invention;

FIG. 11A is a sectional view of a massage roller where the vibrational amplitude is controlled via a cone clutch that engages or disengages from an additional weight with the rotating shaft to change the amplitude of vibration, along the same line as FIG. 4;

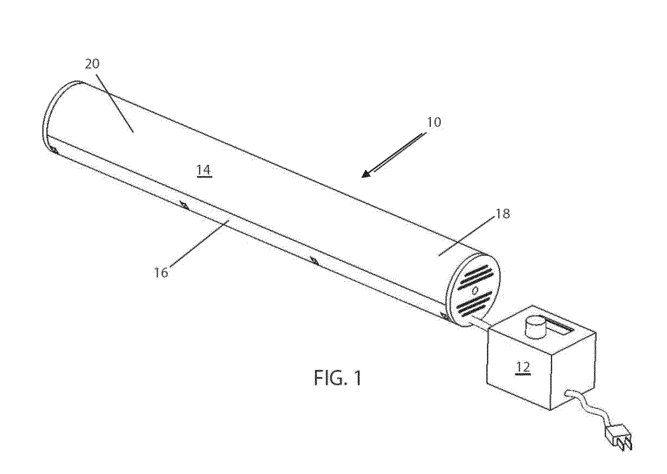

FIG. 1 lB is a detail view of the clutch mechanism according to an embodiment of the invention;

FIG. 12A is a sectional view of a massage roller where the vibrational amplitude is controlled via a two independent sets of flexible cables that changes the eccentricity of two independent sets of weights, along the same line as FIG. 4;

FIG. 12B is a detail view of FIG. 12A showing the two sets of flexible cable feeding though the hollow portion of the shaft toward the two independent sets of weights according to an embodiment of the invention;

FIGS. 13A-13C illustrate a roller where the phases of the outer weights are manually changed with respect to the inboard weights according to an embodiment of the invention; and

FIG. 14 is a perspective view of a weight that is non-spherical, and the circumferential direction is the largest dimension, thereby maximizing the radial eccentricity of the weight according to an embodiment of the invention.

### DESCRIPTION OF THE PREFERRED EMBODIMENTS

The present invention has utility as a vibratory massage roller with variable vibratory amplitude control to facilitate muscle relaxation in a subject. Representative applications of the present invention include massage, physical therapy, yoga, physical conditioning, and general well-being.

In view of the foregoing disadvantages inherent in the known types of massagers, this invention provides a more efficient process for massaging a subject through resort to a vibrational roller with controllable vibrational amplitude having a casing with a casing surface extending over a length of the vibratory roller, and a motor in the housing that is electrically coupled to a power source. The power source is energized to induce user controllable vibratory amplitude to the surface that varies less than thirty amplitude percent across the central seventy percent of the length of the roller. In some embodiments, the vibratory amplitude is controlled to between five and twenty five amplitude percent across the central seventy percent of the length of the roller at operational speeds for subject massage. In still other embodiments, the vibratory amplitude is controlled to within ten amplitude percent across the central seventy percent of the length of the roller at operational speeds for subject massage.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

With reference now to the drawing, the preferred embodiment of the vibrational roller is herein described. It should be noted that the articles “a”, “an”, and “the”, as used in this specification, include plural referents unless the content clearly dictates otherwise.

The present invention represents a departure from the prior art in that the process of massage is enhanced by contacting an energized massaging roller of the present invention with a subject that allows for more even distribution of vibrations, where the amplitude of the vibrations are controlled by the user. In certain embodiments, this is accomplished by utilizing a plurality of eccentric weights and supports that are positioned at different points along a shaft parallel to the axis of the cylinder and are synchronized and positioned for maximum vibrational efficiency. In order to control the amplitude of the vibration, the radial distance of the eccentric weights from the axis of the cylinder is varied. By utilizing a plurality of strategically placed eccentric weights and strategically placed supports, the vibrations engage in reinforcing behavior as they travel the length of the roller, which keeps vibrations uniform throughout the roller, even in a longer roller. By using one motor and one axle, there are fewer parts and less chance of malfunction and is easier to replace or fix when malfunction does occur. Two synchronized, cooperating motors may be used in an alternate embodiment of the invention. The roller may be made in various sizes. Roller length typically is from 12 to 36 inches and circumference may vary from 3 to 10 inches. The roller should be able to resist up to 350 pounds of load. In particular embodiments that include eccentric weights, individual weights may be separate pieces with the weighted portion at a radial distance that is varied in response to one or more user controls with the central rotational shaft. Additionally, it is appreciated to any of the embodiments of the present invention; a thermoelectric resistive heater is readily added to introduce heat and vibration to muscle tissue that is being treated, either through incorporation into the casing or a covering sleeve. Likewise a cooling system is also readily provided a chemical cold pack such as those based on urea, a cool pack based on freeze/thaw cycling; or a thermoelectric Peltier refrigerator, either through incorporation into the casing or a covering sleeve.

In a certain embodiment, twenty five gram eccentric weights or masses are offset up to one inch from the rotating drive shaft 32 that provides uniform vibration from 20-50 Hertz (Hz). Continuing with the specific embodiment, the tube has a three inch to 3.5 inch diameter. Increasing tube diameter will further improve uniformity of vibration. This is especially true when the roller has a length of twenty four inches or longer with a wall thickness of 0.05 to 0.25 inches for a thermoplastic casing. In other embodiments of the present invention, the tube diameters are 3.5 inches to 10 inches. In certain embodiments, a 0.125 to 3 inch thick rubber sleeve is placed over the hard tube casing. The rubber sleeve is readily formed of materials that illustratively include polynorborene, vinyl rubbers, natural rubbers, and foamed versions of any of the aforementioned rubbers. In certain embodiments, an eleven inch drive shaft that is 6-7 mm in diameter is used and is formed of steel.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

An embodiment of an inventive roller shown generally in FIGS. 1-4 at 10. The roller 10 has tubular casing portions 14 and 16 that define a hollow center. It is appreciated that the casing portions 14 and 16 as shown are symmetric and define casing halves; however, in other embodiments one casing portion defines 270 to 340 degrees of the radial distance around the center, with the other casing portion acting as an access door to the center volume. While the tubular casing is depicted herein as a right cylinder, it is appreciated that other cross-sectional shapes are operative herein that include oval, triangular, square, pentagonal, hexagonal and higher polygonal shapes. Resident inside the center, is a shaft (synonymously referred to herein as an “axle”) 32 mounted between a thrust bearing 26 and a motor 24. A coupling may be used to couple the shaft 32 to the motor 24 or the shaft 32 may connect directly to the motor 24 as shown in the figures. Supports 28 are provided to provide linkage between the casing portions 14 and 16 and the shaft 32. Bearings 34 provide a rolling surface, and thus reduced friction, to the shaft 32. A plurality of eccentric weights 30 are positioned along the shaft 32, where the plurality of eccentric weights are positioned at a variable user controlled radial distance from the shaft 32 so as to control the amplitude of vibration. These weights 30 are each a different length from the motor 24, or from a chosen reference point that is on the shaft 32. The weights 30 are eccentrically mounted, meaning that their center of mass is not positioned on the shaft 32 itself, but rather radially displaced some distance, x, away from the axis of rotation of the shaft 32, where the distance x defines the amplitude of the vibration. Thus, when the shaft 32 rotates along its axis, the weights 30 circumscribe a circular motion about the axis and impart a wobble to the shaft 32. This wobble is transmitted to the casing portions 14 and 16 through the supports 28. When a sufficient rotational speed measured for example in revolutions per minute (RPM) are reached, the wobble causes a strong vibration transmitted throughout the casing portions 14 and 16. The weights 30 and supports 28 are strategically positioned about the shaft 32 so as to provide maximum synchronous and uniform vibration advantage during rotation, which is to say the weights 30 are positioned in a manner to provide a uniform vibrational profile throughout the shaft 32 and entire roller 10. The positioning of the weights and supports is of paramount importance as these structures actually impart the uniform vibrational profile to the surface of the roller. Through proper positioning of these weights and supports, vibrations at the surface of the roller will have uniform strength along the length of the roller, with no “dead” or “weak” spots where vibration is not present due to destructive wave interference. The supports 28 and the weights 30 are shown in FIGS. 1-4 with each weight being distal to an adjacent support 28; however, it is appreciated that the relative spacing between a support 28 and the distal weight 30 and indeed, the mass and radial displacement of a given weight are amenable to adjustment to achieve a vibratory amplitude to the surface that varies less than thirty amplitude percent across the central seventy percent of the length of the roller.

Control of the motor 24, and thus the vibration, is achieved through control unit 12, which may be a separate unit as shown in the figures or positioned on the forward end cap 18, which seals one end of the roller 10. The other end is sealed by a second end cap 20. Control unit 12 may have different switches to alter the vibrational characteristics by adjusting the rotational characteristics of the motor 24, as well as the amplitude of the vibration through the radial position of the weights. Such alterations are principally through control of motor rotational speed to set up higher harmonics of vibration that modify the frequency and amplitude of the vibration imparted to a subject in contact with an inventive roller surface. Communication of changes may be displayed on an LCD screen or through indicia as to setting of for example “off”, “low” and “high”.

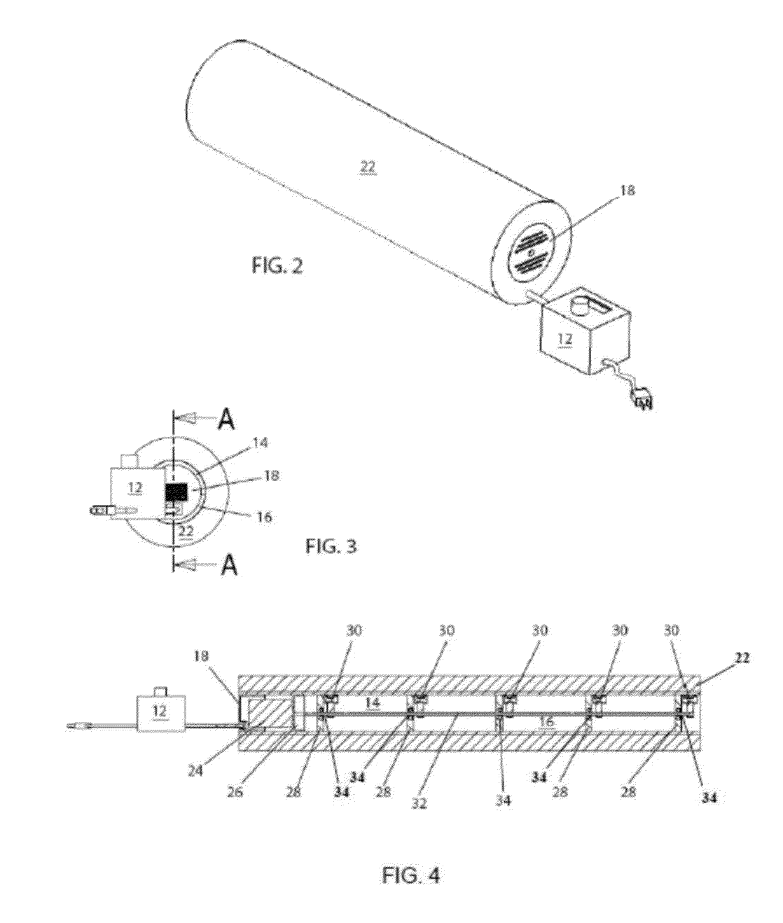

In use, about the casing portions 14 and 16 is an exterior sleeve 22 (FIG. 2) made of a durable, yet deformable material, such as foam rubber or cloth so as to impart a pleasing surface which will efficiently and effectively transmit vibrations and to provide impact resistance, sound dampening, and electromagnetic insulation. As used herein, the term “exterior sleeve” is intended to encompass conventional layers overlaid onto the casing that are either permanent or replaceable overlayers. It is appreciated that multiple such sleeves, each of like or varying material is used simultaneously. An effective layer of such material should be between 0.25 and 3 inches thick, depending upon the size of the roller and internal vibrational motor. The tube may be inserted in padded sleeves of varying textures, density and softness for desired effect on vibration or sensation. Sleeve thickness will be between 1 and 3 inches, depending upon desired effect and materials. This will then impart 2 to 6 inches to the diameter of the roller. The use of sleeves is preferable as the sleeves may be made to be washable, an important feature in clinical use, and can provide protection of the roller unit from elements and wear and tear. Individual sleeves may also be provided for varying textures, support, and firmness and also can be used to provide thermal variation for therapeutic use. It is appreciated that the firmer the rubber sleeve, the more deep tissue massage. It is also appreciated that the sleeve is readily wrapped around the casing and attached thereto through hook and loop fasteners, snapping fittings, a zipper or a pressure fit.

A number of embodiments are possible to achieve a desired vibratory amplitude at the surface that varies less than thirty amplitude percent across the central seventy percent of the length of the roller. It is noted that in several of the embodiments of massage rollers that are described below, the weights or masses 30 are depicted as spheres for simplicity of presentation, however in certain embodiments the weight 30 is non-spherical, and the weight has an actual shape where the circumferential direction will be the largest, thereby maximizing the radial eccentricity of the mass as shown in FIG. 14.

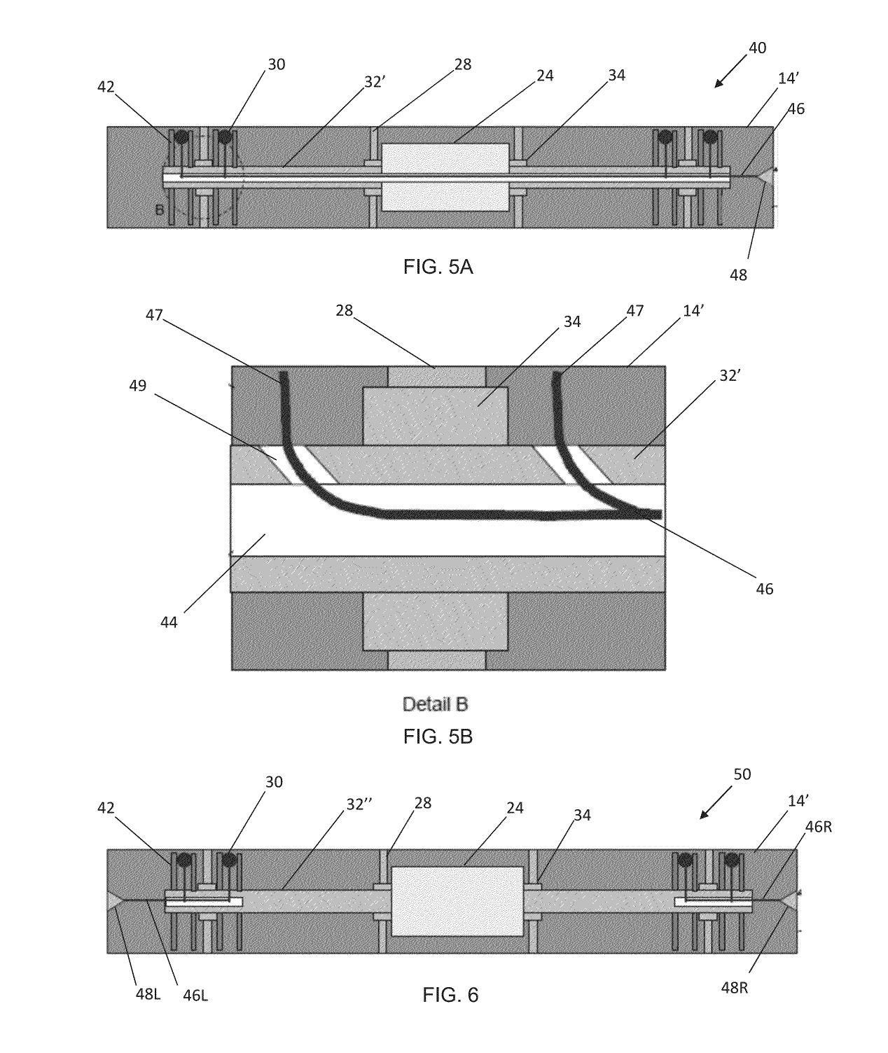

FIGS. 5A and 5B illustrate an embodiment of a message roller 40 that is configured for a user to control the amplitude of the vibration, where the eccentricity of the weights 30 are changed and controlled with a flexible cable 46. The eccentricity of the weights 30 with respect to the radial distance from the shaft 32′ is controlled with the flexible cable 46. Each of the weights 30 of message roller 40 are connected to individual ends 47 extending from the flexible cable 46, where the flexible cable 46 is routed through a hollow center 44 of the rotating shaft 32′, and the cable ends 47 extend through holes 49 in the shaft 32′. The flexible cable 46 terminates at an end of the roller 40, where the flexible cable 46 may be pulled to reduce the eccentricity of the weights 30. The flexible cable 46 rotates with the shaft 32′ and the weights 30, and to accommodate the rotational motion the termination of the flexible cable 46 is with a swivel fitting 48 that allows for rotation and axial translation. Each of the weights 30 are free to ride in guides 42 that provide a channel and restrain the weights 30 in all directions except for radial translation. The guides 42 may be a rigid disk with a center hole that accommodates the bearings 34 that provide a rolling surface, and thus reduced friction, to the rotating shaft 32′. In a similar manner, bulkheads 28 also accommodate the bearings 34 for the shaft 32′, as well as containing the motor 24. In operation as the shaft 32′ rotates, centrifugal force pulls the weights 30 outward in a radial direction away from the shaft 32′, and pulling the flexible cable 46 reduces the redial distance between the weight 30 and the shaft 32′, and thereby acts to reduce the amplitude of vibration.

In a similar manner to the single adjustment flexible cable of FIGS. 5A and 5B, in FIG. 6, in an embodiment of the roller 50, the user controls the amplitude of the vibration of the roller 50 with a right and left side (48R, 48L) swivel fittings that are connected to flexible cables 46R and 46L, respectively. The user pulls the flexible cables (46R, 46L) from both the right and left sides of the roller 50 to control the amplitude of vibration.

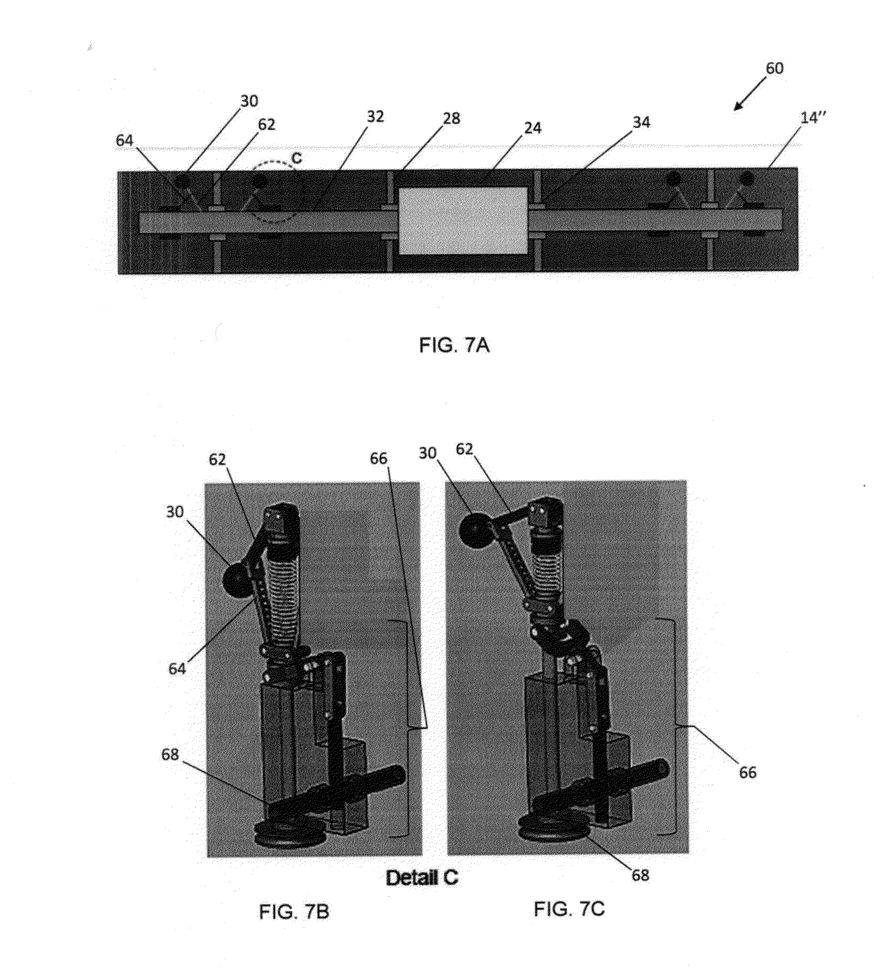

FIGS. 7A-7C illustrate a roller 60 with an umbrella mechanism 66 that is used to control the amplitude of vibration of the roller 60. A user would turn a control button (not shown) that turns a control rod 68 that raises or lowers the umbrella mechanism 66. The raising of the umbrella mechanism 66, which has a series of linkages, draws the fork 64 downward that in turn pulls the pivoting arm 62 inward and lowers the weight 30. The lowering of the weight 30 decreases the radial distance between the weight 30 and the shaft 32, and decreases the amplitude of the vibration of the roller 60. Conversely, the lowering of the umbrella mechanism 66, raises the fork 64 that in turn pushes the pivoting arm 62 outward and raises the weight 30. The raising of the weight 30 increases the radial distance between the weight 30 and the shaft 32, and increases the amplitude of the vibration of the roller 60.

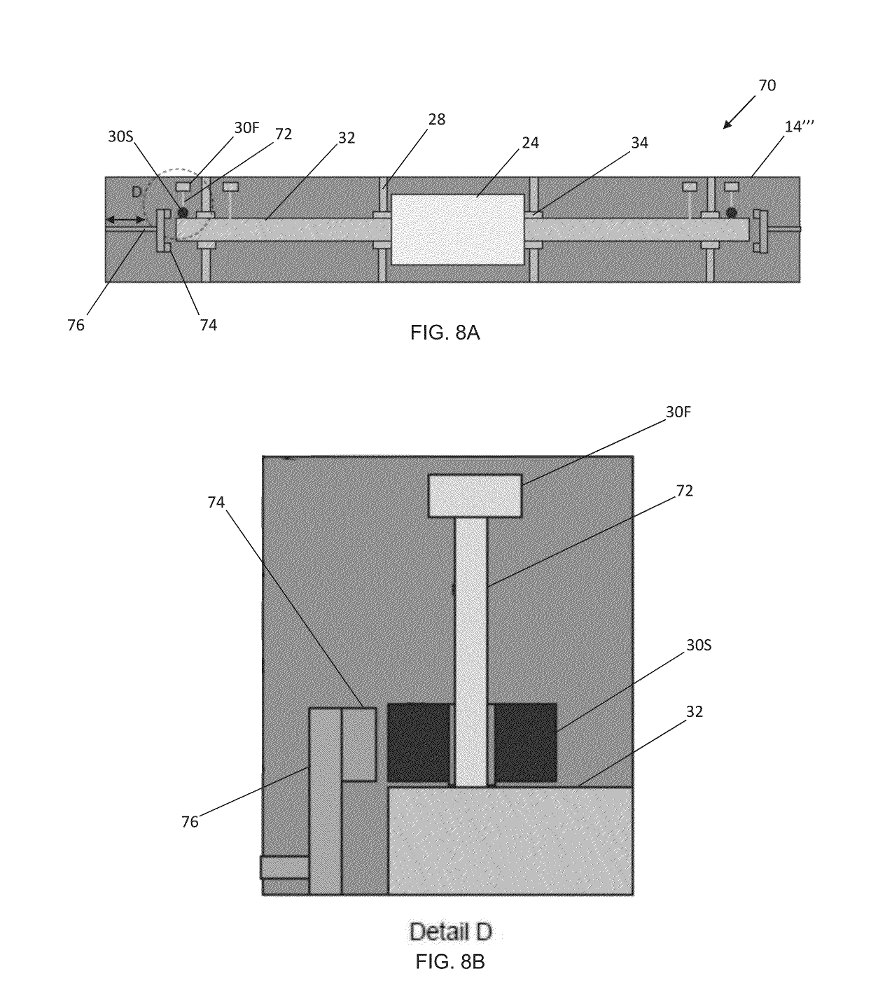

FIG. 8A is a sectional view of a massage roller 70 where the vibrational amplitude is controlled via magnets 74 that control the radial distribution of the total weight that changes the eccentricity of the weights. A fixed weight 30F is attached to the shaft 32 with a rigid rod 72 that accommodates a sliding weight 30S. The sliding weight 30S is generally pulled outward toward the fixed weight 30F by centripetal force pulls the weights 30S outward in a radial direction away from the shaft 32. With the sliding weight 30S positioned against the fixed weight 30F the maximum amplitude of vibration is realized. This maximum amplitude level is realized when the circular washer shaped magnet 74 that is mounted on control arm 76 is retracted away (“open”) from the sliding weight 30S. In order to realize a lower amplitude of vibration, the magnet is moved to a “close” position such that the magnet attracts the sliding weight 30S, and has a great enough pull to overcome the centripetal force on the sliding weight 30S. In general the magnet is moved into the “closed” position with the motor 24 off. FIG. 8B is a detail view of the magnet 74 and sliding weight 30S according to an embodiment of the invention.

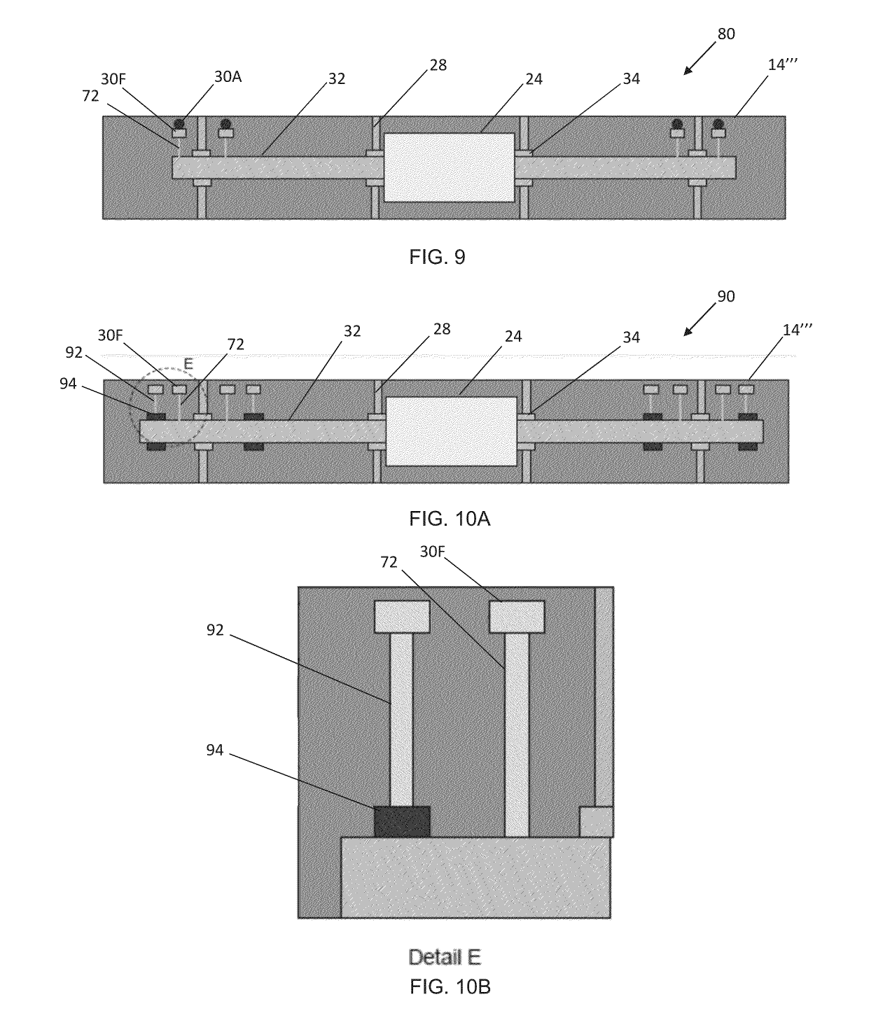

FIG. 9 is a sectional view of a massage roller 80 where the vibrational amplitude is controlled via the manual addition of weight 30A to the eccentricity of fixed weights 30F. The fixed weights 30F are attached to the shaft 32 via rigid rod 72. The added weights 30A may be magnets. In a specific embodiment the added weights 30A may be added through access doors (not shown) in the casing portions 14′″ and 16′″ (not shown).

FIG. 10A is a sectional view of a massage roller 90 where the vibrational amplitude is controlled with a “tunable mass” 92 using an overrunning bearing 94. The massage roller 90 has a fixed weight 30F that is attached to the rotating shaft 32 via rigid rod 72. The tunable mass 92 is attached to the shaft 32 via the overrunning bearing 94. An overrunning bearing is a bearing that allows torque transfer in one direction only. In the message roller 90, the motor 24 can be spun in two directions to achieve two different weight or mass configurations. In the event the motor 24 is run with a clockwise rotation only the fixed weight 30F spins, while a counterclockwise rotation cause both the tunable mass 92 and the fixed weight 30F to spin. FIG. 10B is a detail view of the tunable mass” 92 using an overrunning bearing 94 according to an embodiment of the invention.

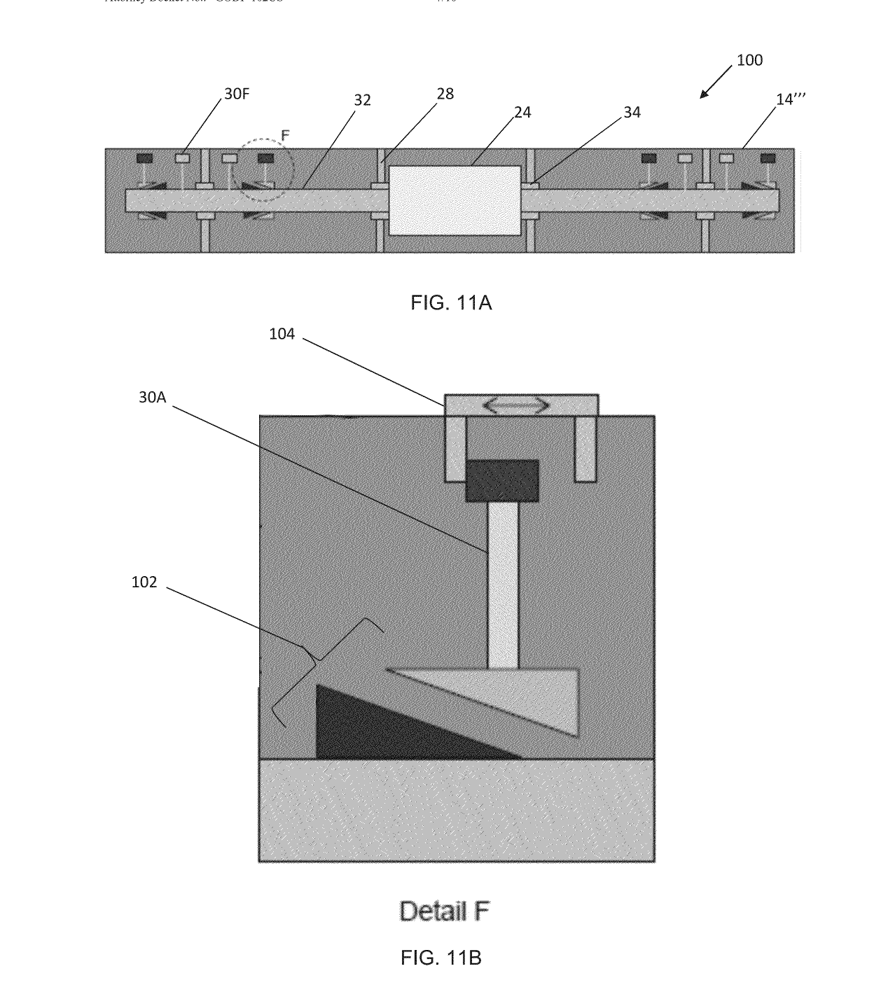

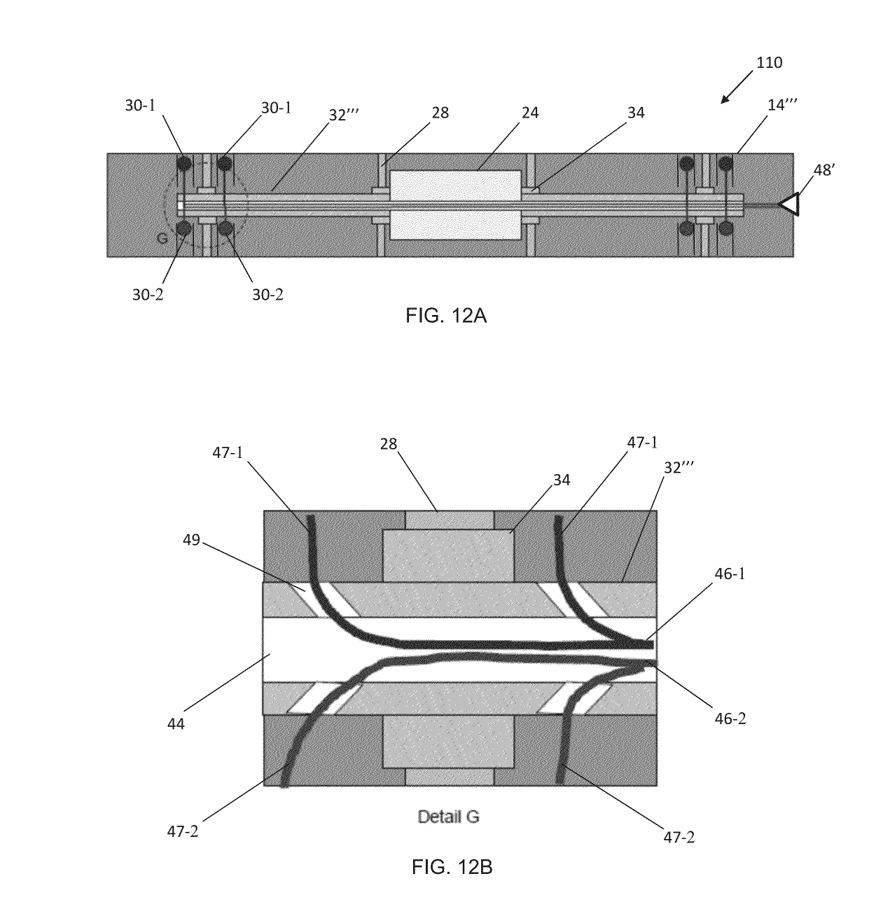

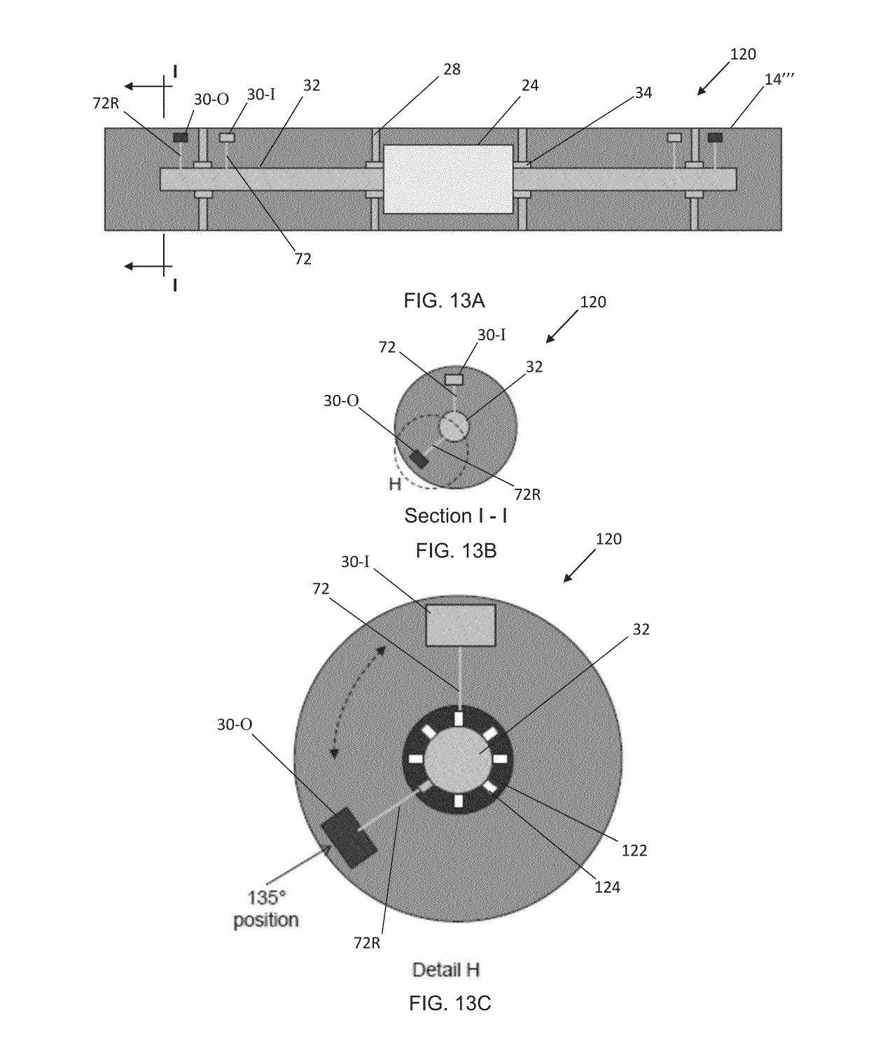

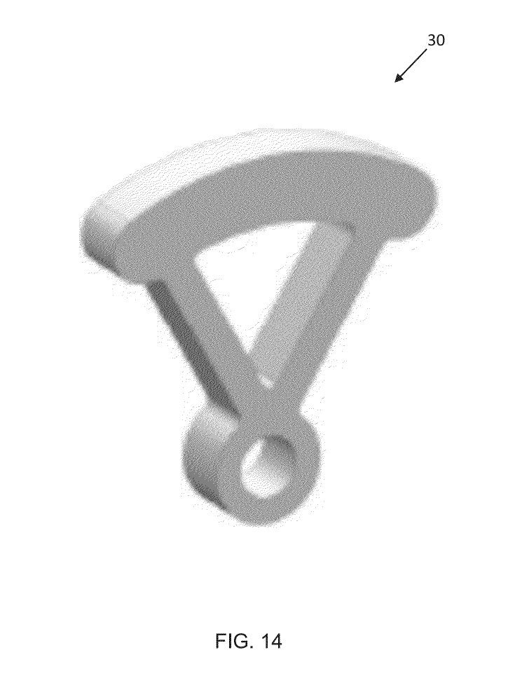

FIG. 11A is a sectional view of a message roller 100 where an additional weight or mass 30A can be added or removed from the rotating shaft 32 with a cone clutch mechanism 102. As can be seen in FIG. 11B, an engagement sleeve or slide switch 104 engages or disengages the addition weight 30A with the cone clutch mechanism 102. When the additional weight 30A is engaged to the shaft 32 with the clutch 102, the amplitude of vibration is maximized.

FIGS. 12A and 12B illustrate an embodiment of a message roller 110 that is configured for a user to control the amplitude of the roller vibration, where the eccentricity of two independent sets of weights 30-1 and 30-2 are changed and controlled with separate independent flexible cables 46-1 and 46-2, respectively. The eccentricity of the two sets of weights 30-1 and 30-2 with respect to the radial distance from the shaft 32′″ is controlled with the flexible cables 46-1 and 46-2, respectively. Each of the set of weights 30-1 and 30-2 of message roller 110 are connected to individual ends 47-1 and 47-2 extending from the flexible cable 46-1 and 46-2, respectively, where the flexible cables 46-1 and 46-2 are routed through a hollow center 44 of the rotating shaft 32″, and the cable ends 47-1 and 47-2 extend through holes 49 in the shaft 32′″. The separate independent flexible cables 46-1 and 46-2 terminate at an end of the roller 110, where the flexible cables 46-1 and 46-2 may be pulled to reduce the eccentricity of the weights 30-1 and 30-2. The flexible cables 46-1 and 46-2 rotate with the shaft 32′″ and the sets of weights 30-1 and 30-2, and to accommodate the rotational motion the termination of the flexible cables 46-1 and 46-2 is with a swivel fitting 48′ that allows for rotation and axial translation. Each of the sets of weights 30-1 and 30-2 are free to ride in guides 42 that provide a channel and restrain the weights 30-1 and 30-2 in all directions except for radial translation. The guides 42 may be a rigid disk with a center hole that accommodates the bearings 34 that provide a rolling surface, and thus reduced friction, to the rotating shaft 32′″. In a similar manner, bulkheads 28 also accommodate the bearings 34 for the shaft 32′″, as well as containing the motor 24. In operation as the shaft 32″′ rotates, centrifugal force pulls the weight sets 30-1 and 30-2 outward in a radial direction away from the shaft 32′″, and pulling the independent flexible cables 46-1 and 46-2 reduces the redial distance between each of the weight sets 30-1 and 30-2 and the shaft 32′″, and thereby acts to reduce the amplitude of vibration. In a specific embodiment, the cable ends 47-1 and 47-2 and corresponding weight sets 30-1 and 30-2 are positioned with a ninety degree offset between the sets (Note: FIGS. 12A and 12B show the position between the weight sets as 180 degrees for simplicity). The relative radial eccentricity of the two weight sets (30-1, 30-2) is tuned to control the effective weight imbalance and the resultant amplitude vibration of the roller 110. A minimum eccentricity configuration has both weight sets held in their inboard position. A medium eccentricity allows one weight set to go to its outboard position. A maximum eccentricity configuration has both weight sets at their outboard position.

FIGS. 13A-13C illustrate a roller 120 where the phases of the removable outer weights 30-O are manually changed with respect to the inboard weights 30-I according to an embodiment of the invention. The inboard weights 30-I are connected to rotating shaft 32 via rigid rod 72. The outer weights 30-O have a removable rigid rod 72R that engages one of a series of slots 124 that are spaced in angular increments on a mounting ring 122 that is fixedly attached to the rotating shaft 32. A user can choose the angular offset for the outer weights 30-O by placing removable rigid rod 72R in one of the series of slots 124. In the example embodiment shown in FIG. 13C, the slots 124 are placed in increments of forty five degrees about the mounting ring 122, and the outer weight 30-O is placed at a 135 degree offset.

Although the present invention has been described with reference to preferred embodiments, numerous modifications and variations can be made and still the result will come within the scope of the invention. No limitation with respect to the specific embodiments disclosed herein is intended or should be inferred.

1. A vibratory massage roller, said massage roller comprising:
a casing having a surface extending over a length of said vibratory roller, a motor in said housing, said motor electrically coupled to a power source;
one or more weights within said casing, where the configuration of the positions of said one or more weights in relation to a rotating shaft concentric to said casing and driven by said motor determines a magnitude of a vibratory amplitude; and
wherein said power source induces a vibratory amplitude to the surface that varies less than thirty amplitude percent across the central seventy percent of the length.

2. The roller of claim 1 further comprising a foam or rubber sleeve around said casing.

3. The roller of claim 1 further comprising a flexible cable routed through said shaft, said flexible cable having one or more individual ends, where each individual end is attached to one of said one or more weights, where pulling said flexible cable reduces a radial distance between said one or more weights and said shaft, and thereby acts to reduce the magnitude of vibratory amplitude.

4. The roller of claim 3, wherein said flexible cable terminates in a swivel fitting that allows for rotation and axial translation of said flexible cable.

5. The roller of claim 3 further comprising a second independent flexible cable with a second set of one or more ends attached to a second set of one or more weights, where pulling said second flexible cable reduces a second radial distance between said second set of one or more weights and said shaft.

6. The roller of claim 5 wherein said flexible cable and said second flexible cable both terminate on a first end of said massage roller.

7. The roller of claim 5 wherein said flexible cable and said second flexible cable terminate on a first end and a second end, respectively, of said massage roller.

8. The roller of claim 1 further comprising one or more umbrella mechanisms for the adjustment of said one or more weights, where each of said umbrella mechanisms comprise a series of linkages that draws a fork downward with the raising of said umbrella mechanism that in turn pulls a pivoting arm inward attached to one of said one or more weights and lowers said one of said one or more weights where the lowering of the one weight decreases a radial distance between the one weight and said shaft, and decreases the magnitude of the vibratory amplitude of said roller, and conversely, the lowering of said umbrella mechanism, raises the fork that in turn pushes the pivoting arm outward and raises said one weight, the raising of the one weight increases the radial distance between the one weight and said shaft, and increases the amplitude of the vibration of said roller.

9. The roller of claim 1 further comprising one or more sliding weights with a radial position with respect to said shaft controlled by one or more magnets.

10. The roller of claim 1 further comprising one or more overrunning bearings that selectively join one or more tunable masses to said rotating shaft depending on a direction of rotation of said shaft.

11. The roller of claim 1 further comprising a cone clutch mechanism selectively joining said one or more weights to said shaft.

12. A process of massaging a subject comprising:
providing a vibratory massage roller, said massage roller comprising: a casing having a surface extending over a length of said vibratory roller, a motor in said housing, said motor electrically coupled to a power source;
configuring one or more weights within said casing, where the positions of said one or more weights in relation to a rotating shaft concentric to said casing and driven by said motor determines a magnitude of a vibratory amplitude;
energizing said power source to induce a vibratory amplitude to the surface that varies less than thirty amplitude percent across the central seventy percent of the length; and
contacting said massage roller with the subject to massage the subject.

13. The process of claim 12 further comprising heating or cooling the massage roller.

14. The process of claim 12 further comprising placing a foam or rubber sleeve around said casing.

15. The process of claim 12 further comprising a flexible cable routed through said shaft, said flexible cable having one or more individual ends, where each end is attached to one of said one or more weights, where pulling said flexible cable reduces a redial distance between said one or more weights and said shaft, and thereby acts to reduce the magnitude of vibratory amplitude.

16. The process of claim 15, wherein said flexible cable terminates in a swivel fitting that allows for rotation and axial translation of said flexible cable.

17. The process of claim 16 further comprising a second independent flexible cable with a second set of one or more ends attached to a second set of one or more weights, where pulling said second flexible cable reduces a second redial distance between said second set of one or more weights and said shaft.

18. The process of claim 17, wherein said flexible cable and said second flexible cable both terminate on a first end of said massage roller.

19. The process of claim 17 wherein said flexible cable and said second flexible cable terminate on a first end and a second end, respectively, of said massage roller.

20. The process of claim 12 further comprising one or more umbrella mechanisms for said configuring of said one or more weights, where each of said umbrella mechanisms comprise a series of linkages that draws a fork downward with the raising of said umbrella mechanism that in turn pulls a pivoting arm inward attached to one of said one or more weights and lowers said one of said one or more weights, where the lowering of the one weight decreases a radial distance between the one weight and said shaft, and decreases the magnitude of the vibratory amplitude of said roller, and conversely, the lowering of said umbrella mechanism, raises the fork that in turn pushes the pivoting arm outward and raises said one weight, the raising of the one weight increases the radial distance between the one weight and said shaft, and increases the amplitude of the vibration of said roller.

21. The process of claim 12 further comprises one or more sliding weights where said configuring further comprises adjusting a radial position of said one or more sliding weights with respect to said shaft with one or more magnets.

22. The process of claim 12 wherein said configuring further comprises the manual addition of one or more additional weights.

23. The process of claim 12 further comprising one or more overrunning bearings that selectively join one or more tunable masses to said rotating shaft depending on a direction of rotation of said shaft.

24. The process of claim 23 further comprising a cone clutch mechanism, where said configuring further comprises selectively joining said one or more weights to said shaft with said cone clutch mechanism.
